(19) United States
(12) Patent Application Publication
(10) Pub. No.: US 2014/0378830 A1
(43) Pub. Date: Dec. 25, 2014

(54) Method for stimulating a brain to form a cognitive reaction image

(71) Applicant: Ming-Hsin Li, Taoyuan County (TW)

(72) Inventor: Ming-Hsin Li, Taoyuan County (TW)

(21) Appl. No.: 13/925,806

(22) Filed: Jun. 24, 2013

(51) Int. Cl. A61B 6/00; A61B 6/03

(52) U.S. Cl. CPC A61B 6/501; A61B 6/037
USPC 600/431

(57) ABSTRACT

A method for stimulating a brain to form a cognitive reaction image is provided, which includes administrating ¹¹C-isotope labeled glucose to a test recipient closing eyes, exposing the test recipient to visual stimulus, auditory stimulus and cognitive stimulus, respectively, and then monitoring the test recipient by a brain imaging device and collecting brain imaging data to compare variation between actions of the administrated ¹¹C-isotope labeled glucose in the test recipient's brain before and after the test recipient being exposed to the stimulus. The brain images in correspondence with the brain reacting to the visual, auditory and cognitive stimuli are obtained by detecting the metabolic process of the ¹¹C-isotope labeled glucose in brain.

### FIELD OF THE INVENTION

The present invention is related to a method for stimulating a brain to form a cognitive reaction images, and especially to a method for stimulating a brain to form a cognitive reaction images by detecting the actions of ¹¹C-isotope labeled glucose (such as [1-C-11]-2-deoxy-D-glucose) in brain.

### DESCRIPTION OF RELATED ART

The brain of mammals is the most important part of the central nervous system (CNS) and in charge of mind, thinking, somatic sense, auditory sense, visual sense, as well as producing psychological phenomena. The human brain has the forebrain, midbrain, hindbrain and spinal cord. The forebrain consists of the cerebral cortex, the limbic lobes and the brain stem. The cerebral cortex has three parts: the frontal lobe, the temporal lobe and the occipital lobe. Conventionally, electroencephalography (EEG), magnetoencephalography (MEG), positron emission tomography (PET), single photon emission tomography (SPECT), functional magnetic resonance imaging (fMRI) are used for brain imaging to analyze the function area in the brain responsive to cognitive reactions.

In general, most of brain scanning technology for obtaining brain images anticipate that the changes of hemodynamic response (HR) caused by stimulation in experiments or relative incidents may occur in the net formed by several parts of the brain. Such analytic approaches indicates that our knowledge of the brain is confined in a linear system, that is, the definition of “voxel” is over-simplified according to the construction of brain modularity, which states that “different brain areas responsive are to different cognitive functions.” and asserts that the HR noises and non-HR noises in the brain images have homogeneity in different brain areas on different incidents. However, with more understanding of the complexity of brain, researchers eventually realize that the change of HR is unpredictable and the brain function is extremely complex.

As for fMRI and PET scanning, both of the two imaging methods have high spatial resolution but low time resolution. Further, EEG and MEG can measure the distributions of the magnetic field and the electric field caused by the flow of ions when the nerve tissue is activated, and the two methods have excellent millisecond-level time resolution. EEG and MEG can be applied in the research of mechanism of the neuronal activities in the brain network. Transcranial magnetic stimulation (TMS) requires more development for application of modulating the plasticity of neural network. Yet, such technologies like EEG, MEG and TMS do not have high spatial resolution. Since the approaches described above are based on different medical physics mechanism, the properties of the resulting images and signal information are also varied.

As for radiopharmaceutical-assisted brain imaging, the mostly used drug is ¹⁸F isotope labeled drugs, for example, [F-18]-2-deoxyglucose (F-18-FDG). The chemical structure of F-18-FDG is similar to glucose. The distribution of F-18-FDG in a living body depends on the metabolism of glucose in tissues and organisms. The images from PET scanning can illustrate the physiological activities of glucose in a human body, and diagnosis can be achieved through quantification of the uptake of glucose with reference to a standard uptake volume (SUV). If tumor cells divide and proliferate quickly and abnormally increase the metabolic rate of normal cells through regulating genes and proteins, the PET images of F-18-FDG in tumor organisms show higher uptake volume than SUV.

Nevertheless, the halftime of F-18-FDG is up to 109 minutes, and it is too long for the measurement of brain psychological responses, behaviors, cognitions. Therefore, besides F-18-FDG, developing new drugs for application in brain imaging in order to break through the bottlenecks of conventional approaches is necessary and urgent in this technical field.

### SUMMARY OF THE INVENTION

The primary objective of the present invention is to use collected brain imaging data and reconstructed images by a brain imaging device for comparison of changes in metabolic rate of C-11-2DG glucose in the brain of a test recipient for further research in the field of radiopharmaceutical development and brain imaging for analysis of brain function and psychological neuroscience. Among various kinds of application of nuclear medicine, the technology of the present invention is essential to the cognitive psychology and management strategy.

Another objective of the present invention is to provide a method for stimulating a brain to form a cognitive reaction image through monitoring the test recipient by a brain imaging device, obtaining a base brain imaging data from the brain imaging device, using the base brain imaging data as a standard uptake value, and monitoring the test recipient exposed to various cognitive stimulus by the brain imaging device and obtaining various brain imaging data in correspondence with the various cognitive stimulus from the brain imaging device, and comparing the various brain imaging data with the base brain imaging data to observe the variation between the actions of the test recipient before and after being exposed to the cognitive stimulus.

Another objective of the present invention is to use an isotope labeled pharmaceuticals as an imaging agent and administrate into a test recipient's body. Annihilation reaction can occur when released positrons of the isotopes react with electrons in cells of the test recipient's body, and pair of 511 KeV gamma rays in opposite direction is formed, wherein the resulting gamma rays can be detected by a positron imaging device and the raw data can be calculated to reconstruct images.

### BRIEF DESCRIPTION OF THE DRAWINGS

FIG. 1 is a graph showing the actions of C-11-2DG in the brain of a test recipient before and after being exposed to a visual, auditory and cognitive stimulus, respectively.

### DETAILED DESCRIPTION OF THE PREFERRED EMBODIMENTS

The present invention provides a method for stimulating a brain to form a cognitive reaction image, and comprising steps of:
(a) making a test recipient close eyes, not to think of anything or any questions;
(b) administrating ¹¹C-isotope labeled glucose to the test recipient;
(c) monitoring the test recipient by a brain imaging device, obtaining a first brain imaging data from said brain imaging device, and using said first brain imaging data as a standard uptake value;
(d) making the test recipient be exposed to a visual stimulus, open eyes and imagine a melody or a rhythm;
(e) administrating said ¹¹C-isotope labeled glucose to the test recipient exposed to said visual stimulus;
(f) monitoring the test recipient exposed to said visual stimulus by a brain imaging device, and obtaining a second brain imaging data from said brain imaging device, wherein said second brain imaging data shows the action of said administrated ¹¹C-isotope labeled glucose in the brain of the test recipient after being exposed to said visual stimulus;
(g) comparing said second brain imaging data with said first brain imaging data to observe the variation between the actions of said administrated ¹¹C-isotope labeled glucose in the brain of the test recipient before and after being exposed to said visual stimulus.

In one embodiment of the present invention, step (a) comprises making a test recipient close eyes in a dark environment with no sound or voice and with no light to let the pupils cannot perceive any image.

In one embodiment of the present invention, step (b) comprises making the test recipient open eyes in an environment which is light-colored or decorated by signs related to music and images of instruments to let the pupils perceive images in such environment.

In a preferred embodiment, said ¹¹C-isotope labeled glucose can include, but not limited to [1-C-11]2-Deoxy-D-Glucose or other alternative substance.

In a preferred embodiment, said visual stimulus can include, but not limited to, a test room filled with a single color or multiple colors, or decorated by signs related to music and images of instruments, or other alternative stimulus.

In a preferred embodiment, said brain imaging device can include, but not limited to, a PET device, an SPECT device, or other alternative devices.

In a preferred embodiment, the melody or the rhythm which the test recipient imagines can be familiar to the test recipient.

In another aspect, the present invention also provides a method for stimulating a brain to form a cognitive reaction image, comprising steps of:
(a′) making a test recipient close eyes, not to think of anything or any questions;
(b′) administrating ¹¹C-isotope labeled glucose to the test recipient;
(c′) monitoring the test recipient by a brain imaging device, obtaining a third brain imaging data from said brain imaging device, and using said third brain imaging data as a standard uptake value;
(d′) making the test recipient be exposed to an auditory stimulus, open eyes and listen to a melody or a rhythm;
(e′) administrating said ¹¹C-isotope labeled glucose to the test recipient exposed to said auditory stimulus;
(f) monitoring the test recipient exposed to said auditory stimulus by said brain imaging device, and obtaining a fourth brain imaging data from said brain imaging device, wherein said second fourth imaging data shows the action of said administrated ¹¹C-isotope labeled glucose in the brain of the test recipient after being exposed to said auditory stimulus;
(g′) comparing said fourth brain imaging data with said third brain imaging data to observe the variation between the actions of said administrated ¹¹C-isotope labeled glucose in the brain of the test recipient before and after being exposed to said auditory stimulus.

In one embodiment of the present invention, step (a′) comprises making a test recipient close eyes in a dark environment with no sound or voice and with no light to let the pupils cannot perceive any image.

In one embodiment of the present invention, step (b′) comprises making the test recipient open eyes and ears (no barrier such as earplugs in ears) in an environment with soft light and music played therein to let the ears perceive voices in such environment.

In a preferred embodiment, said ¹¹C-isotope labeled glucose can include, but not limited to [1-C-11]2-Deoxy-D-Glucose or other alternative substance.

In a preferred embodiment, said auditory stimulus can include, but not limited to, music played via a disk or a tape, or performing live music in front of the test recipient.

In a preferred embodiment, said brain imaging device can include, but not limited to, a PET device, an SPECT device, or other alternative devices.

In a preferred embodiment, the music which the test recipient hears can be familiar to the test recipient.

In yet another aspect, the present invention also provides a method for stimulating a brain to form a cognitive reaction image is provided, and comprising steps of:
(a″) making a test recipient close eyes, not to think of anything or any questions;
(b″) administrating ¹¹C-isotope labeled glucose to the test recipient;
(c″) monitoring the test recipient by a brain imaging device, obtaining a fifth brain imaging data from said brain imaging device, and using said fifth brain imaging data as a standard uptake value;
(d″) making the test recipient be exposed to an cognitive stimulus, open eyes and imagine playing an instrument by himself or moving fingers with a melody or a rhythm;
(e″) administrating said ¹¹C-isotope labeled glucose to the test recipient exposed to said cognitive stimulus;
(f″) monitoring the test recipient exposed to said cognitive stimulus by said brain imaging device, and obtaining a sixth brain imaging data from said brain imaging device, wherein said sixth imaging data shows the action of said administrated ¹¹C-isotope labeled glucose in the brain of the test recipient after being exposed to said cognitive stimulus;
(g″) comparing said sixth brain imaging data with said fifth brain imaging data to observe the variation between the actions of said administrated ¹¹C-isotope labeled glucose in the brain of the test recipient before and after being exposed to said cognitive stimulus.

In one embodiment of the present invention, step (a″) comprises making a test recipient close eyes in a dark environment with no sound or voice and with no light to let the pupils cannot perceive any image.

In one embodiment of the present invention, step (b″) comprises making the test recipient open eyes and ears (no barrier such as earplugs in ears) in an environment with soft light and without sounds or voices, and imagine performing music by himself via moving fingers to imitate movement of playing an instrument or move feet to hit something repeatedly with the beats of music.

In a preferred embodiment, said ¹¹C-isotope labeled glucose can include, but not limited to [1-C-11]2-Deoxy-D-Glucose or other alternative substance.

In a preferred embodiment, said cognitive stimulus can include, but not limited to that the test recipient in a test room imagine a melody or a rhythm, and move fingers to imitate movement of playing an instrument to perform the melody or the rhythm or move feet to hit something repeatedly with the beats of the melody or the rhythm.

In a preferred embodiment, said brain imaging device can include, but not limited to, a PET device, an SPECT device, or other alternative devices.

In a preferred embodiment, the melody or the rhythm which the test recipient imagines can be familiar to the test recipient.

Glucose is the major energy source for brain activities. The metabolism of glucose in the brain can reflect the brain function. Decrease of the metabolic level of glucose indicates that a part of activities of neurons are inhibited, and it is a kind of behavior of brain dysfunction. Glucose analogs, such as [1-C-11]-2-deoxy-D-glucose, can be taken in by high-glucose-using cells. In such cells, phosphorylation will impede glucose releasing with its original form from the cells.

Due to the requirement of the oxygen of 2′ hydroxyl group in normal glucose for subsequent glycolysis, the glucose analogs (such as [1-C-11]-2-deoxy-D-glucose) in the cells cannot be metabolized, and the phosphorylated glucose analogs (such as [1-C-11]-2-deoxy-D-glucose-6-phosphate) also cannot be involved in the glycolytic pathway before radioactive decay. Thus, the distribution of [1-C-11]-2-deoxy-D-glucose can adequately reflect glucose uptake of high-glucose-using cells and the location of phosphorylated glucose in the brain. The metabolic catalysis of [1-C-11]-2-deoxy-D-glucose can be inhibited before decay due to lacking oxygen of the 2′ position in the chemical structure. Through positron radiation, the carbon isotopes, ¹¹C atoms, can decay and change to ¹¹B atoms totally, the half lifetime of ¹¹C atoms changing to ¹¹B atom is about 20 minutes. ¹¹C atoms decay and change to ¹¹B atoms after radioactive decay, and also will not emit a 511 KeV gamma ray.

According to the present invention described above, the objective of the present invention is to provide a method for stimulating a brain via a visual, auditory and cognitive stimulus, and using of [1-C-11]2-Deoxy-D-Glucose as a radiopharmaceutical for monitoring its reaction and localization in a test recipient's brain through its brain imaging. Therefore, the resulting data from brain imaging can be obtained for neuropsychological analysis.

### Examples

Drugs and materials [1-C-11]2-Deoxy-D-Glucose (referred to as C-11-2DG hereinafter) is formulated into an isotonic sterile radiopharmaceutical level injection by a automatic synthesis box from PET Center with radiochemical purity of 99%.

Test Process A
1. A test recipient is made on an empty stomach for 6 hours.
2. The test recipient is made to close eyes, not to think of anything or any questions.
3. Then, C-11-2DG with dosage of 16 to 20 mCi is injected into the test recipient.
4. After injection, the test recipient rests for 45 minutes in a quiet environment, and then urinates. The test recipient lies on an examination bed and monitored via brain imaging using a PET imaging device (such as GE Discovery LS PET scanner). Attenuation correction is applied in the resulting raw data to reconstruct a PET image, Image 1, showing the action of C-11-2DG in the brain of the test recipient and used as a standard uptake value.
5. The test recipient is made to be exposed to a visual stimulus, open eyes and imagine playing an instrument by himself or moving fingers with a melody or a rhythm that was well-known by the test recipient.
6. Then, C-11-2DG with dosage of 16 to 20 mCi is injected into the test recipient.
7. After injection, the test recipient rests for 45 minutes in a quiet environment, and then urinates. The test recipient lies on an examination bed and monitored via brain imaging using a PET imaging device (such as GE Discovery LS PET scanner). Attenuation correction is applied in the resulting raw data to reconstruct a PET image, Image 2, showing the action of C-11-2DG in the brain of the test recipient after being exposed to the visual stimulus.
8. Image 2 is compared with Image 1 to observe the variation between the actions of C-11-2DG in the brain of the test recipient before and after being exposed to the visual stimulus.

Test Process B
1. A test recipient is made on an empty stomach for 6 hours.
2. The test recipient is made to close eyes, not to think of anything or any questions.
3. Then, C-11-2DG with dosage of 16 to 20 mCi is injected into the test recipient.
4. After injection, the test recipient rests for 45 minutes in a quiet environment, and then urinates. The test recipient lies on an examination bed and monitored via brain imaging using a PET imaging device (such as GE Discovery LS PET scanner). Attenuation correction is applied in the resulting raw data to reconstruct a PET image, Image 3, showing the action of C-11-2DG in the brain of the test recipient and used as a standard uptake value.
5. The test recipient is made to be exposed to an auditory stimulus, open eyes and listen to a melody or a rhythm that was well-known by the test recipient.
6. Then, C-11-2DG with dosage of 16 to 20 mCi is injected into the test recipient.
7. After injection, the test recipient rests for 45 minutes in a quiet environment, and then urinates. The test recipient lies on an examination bed and monitored via brain imaging using a PET imaging device (such as GE Discovery LS PET scanner). Attenuation correction is applied in the resulting raw data to reconstruct a PET image, Image 4, showing the action of C-11-2DG in the brain of the test recipient after being exposed to the auditory stimulus.
8. Image 4 is compared with Image 3 to observe the variation between the actions of C-11-2DG in the brain of the test recipient before and after being exposed to the auditory stimulus.

Test Process C
1. A test recipient is made on an empty stomach for 6 hours.
2. The test recipient is made to close eyes, not to think of anything or any questions.
3. Then, C-11-2DG with dosage of 16 to 20 mCi is injected into the test recipient.
4. After injection, the test recipient rests for 45 minutes in a quiet environment, and then urinates. The test recipient lies on an examination bed and is monitored via brain imaging using a PET imaging device (such as GE Discovery LS PET scanner). Attenuation correction is applied in the resulting raw data to reconstruct a PET image, Image 5, showing the action of C-11-2DG in the brain of the test recipient and is used as a standard uptake value.
5. The test recipient is made to be exposed to an cognitive stimulus, open eyes and imagine playing an instrument by himself or moving fingers with a melody or a rhythm that was well-known by the test recipient.
6. Then, C-11-2DG with dosage of 16 to 20 mCi is injected into the test recipient.
7. After injection, the test recipient rests for 45 minutes in a quiet environment, and then urinates. The test recipient lies on an examination bed and monitored via brain imaging using a PET imaging device (such as GE Discovery LS PET scanner). Attenuation correction is applied in the resulting raw data to reconstruct a PET image, Image 6, showing the action of C-11-2DG in the brain of the test recipient after being exposed to the auditory stimulus.
8. Image 6 is compared with Image 5 to observe the variation between the actions of C-11-2DG in the brain of the test recipient before and after being exposed to the cognitive stimulus.

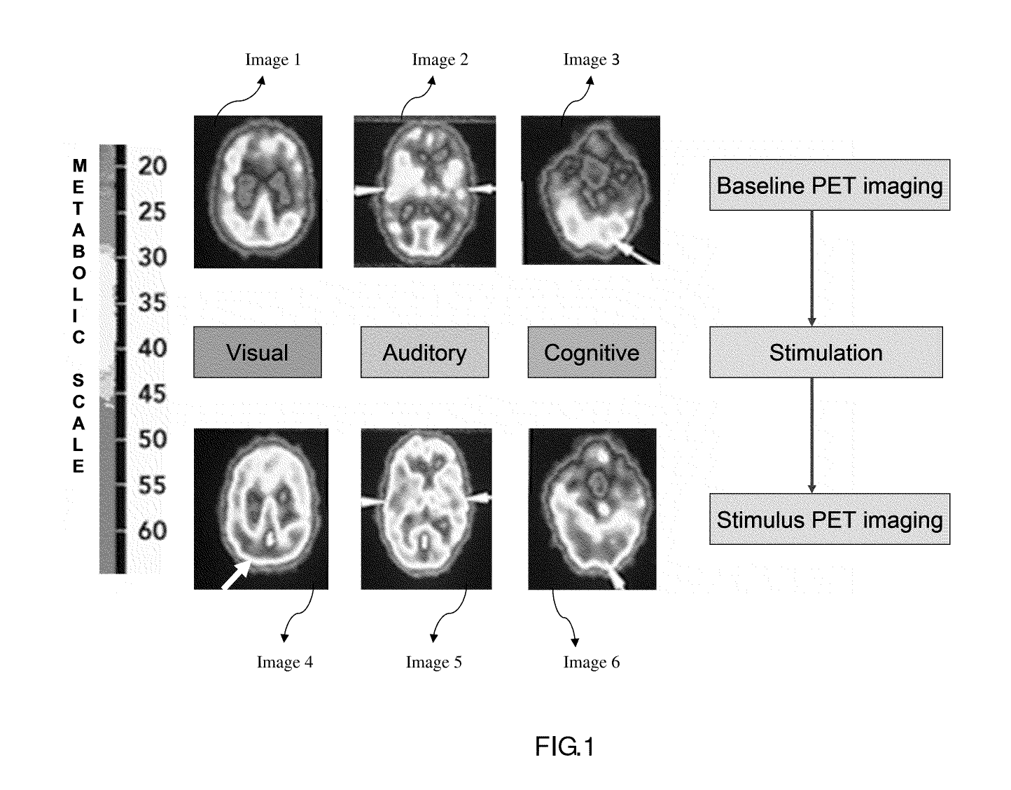

As shown in FIG. 1, it is a graph showing the scales of metabolic changes of C-11-2DG in the brain of a test recipient before and after being exposed to a visual, auditory and cognitive stimulus respectively, including Image 1, Image 2, Image 3, Image 4, Image 5 and Image 6. In the three test processes, the location of C-11-2DG in the brain of the test recipient before and after being exposed to stimuli described above are similar, however, all of the metabolic volumes of C-11-2DG in the brain of the test recipient after being exposed to stimuli are significantly increased.

After being exposed to the visual stimulus, the metabolic volumes of C-11-2DG in the rhombencephalon and the prefrontal are increased. The result of the test process about visual stimulation is demonstrated through the variation between Images1 and Image 2. However, brain images using traditional scanning technology, for example, functional magnetic resonance imaging (fMRI), only able to display domains with increasing rate of blood stream and cannot directly illustrate the changes of activities of the visually stimulated brain.

After being exposed to the auditory stimulus, the metabolic volumes of C-11-2DG in the lateral domain on both side of the brain are increased. The result of the test process about auditory stimulation is demonstrated through the variation between Images3 and Image 4. However, brain images using traditional scanning technology, for example, functional magnetic resonance imaging (fMRI), only able to display domains with increasing rate of blood stream and cannot directly illustrate the changes of activities of the auditorily stimulated brain.

After being exposed to the cognitive stimulus, the metabolic volumes of C-11-2DG in the rhombencephalon are increased. The result of the test process about cognitive stimulation is demonstrated through the variation between Images5 and Image 6. However, brain images using traditional scanning technology, for example, functional magnetic resonance imaging (fMRI), only able to display domains with increasing rate of blood stream and cannot directly illustrate the changes of activities of the cognitively stimulated brain.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for stimulating a brain to form cognitive reaction image, comprising steps of:
making a test recipient close eyes, not to think of anything or any questions;
administrating isotope labeled glucose to the test recipient;
monitoring the test recipient by a brain imaging device,
obtaining a first brain imaging data from said brain imaging device, and using said first brain imaging data as a standard uptake value;
making the test recipient be exposed to a visual stimulus, open eyes and imagine a melody or a rhythm;
administrating said isotope labeled glucose to the test recipient exposed to said visual stimulus;
monitoring the test recipient exposed to said visual stimulus by said brain imaging device, and obtaining a second brain imaging data from said brain imaging device, wherein said second brain imaging data shows the action of said administrated isotope labeled glucose in the brain of the test recipient after being exposed to said visual stimulus;
comparing said second brain imaging data with said first brain imaging data to observe the variations of metabolic rate between the actions of said administrated isotope labeled glucose in the brain of the test recipient before and after being exposed to said visual stimulus.

2. The method as claimed in claim 1, wherein said isotope labeled glucose is [1-C-11]2-Deoxy-D-Glucose.

3. The method as claimed in claim 1, wherein said visual stimulus is a test room filled with a single color or multiple colors, or decorated by signs related to music and images of instruments.

4. The method as claimed in claim 1, wherein said brain imaging device is a PET device or an SPECT device.

5. A method for stimulating a brain to form cognitive reaction image, comprising steps of:
making a test recipient close eyes, not to think of anything or any questions;
administrating ¹¹C-isotope labeled glucose to the test recipient;
monitoring the test recipient by a brain imaging device, obtaining a third brain imaging data from said brain imaging device, and using said third brain imaging data as a standard uptake value;
making the test recipient be exposed to an auditory stimulus, open eyes and listen to a melody or a rhythm;
administrating said ¹¹C-isotope labeled glucose to the test recipient exposed to said auditory stimulus;
monitoring the test recipient exposed to said auditory stimulus by a brain imaging device, and obtaining a fourth brain imaging data from said brain imaging device, wherein said second fourth imaging data shows the action of said administrated ¹¹C-isotope labeled glucose in the brain of the test recipient after being exposed to said auditory stimulus;
comparing said fourth brain imaging data with said third brain imaging data to observe the variations of metabolic rate between the actions of said administrated ¹¹C-isotope labeled glucose in the brain of the test recipient before and after being exposed to said auditory stimulus.

6. The method as claimed in claim 5, wherein said ¹¹C-isotope labeled glucose is [1-C-11]2-Deoxy-D-Glucose.

7. The method as claimed in claim 5, wherein said auditory stimulus is music played via a disk or a tape, or performing live music in front of the test recipient.

8. The method as claimed in claim 5, wherein said brain imaging device is a PET device or an SPECT device.

9. A method for stimulating a brain to form a cognitive reaction image, comprising steps of:
making a test recipient close eyes, not to think of anything or any questions;
administrating ¹¹C-isotope labeled glucose to the test recipient;
monitoring the test recipient by a brain imaging device, obtaining a fifth brain imaging data from said brain imaging device, and using said fifth brain imaging data as a standard uptake value;
making the test recipient be exposed to an cognitive stimulus, open eyes and imagine playing an instrument by himself or moving fingers with a melody or a rhythm;
administrating said ¹¹C-isotope labeled glucose to the test recipient exposed to said cognitive stimulus;
monitoring the test recipient exposed to said cognitive stimulus by a brain imaging device, and obtaining a sixth brain imaging data from said brain imaging device, wherein said sixth imaging data shows the action of said administrated ¹¹C-isotope labeled glucose in the brain of the test recipient after being exposed to said cognitive stimulus;
comparing said sixth brain imaging data with said fifth brain imaging data to observe the variations of metabolic rate between the actions of said administrated ¹¹C-isotope labeled glucose in the brain of the test recipient before and after being exposed to said cognitive stimulus.

10. The method as claimed in claim 9, wherein said ¹¹C-isotope labeled glucose is [1-C-11]2-Deoxy-D-Glucose.

11. The method as claimed in claim 9, wherein said cognitive stimulus is that the test recipient in a test room imagine a melody or a rhythm, and move fingers to imitate movement of playing an instrument to perform the melody or the rhythm or move feet to hit something repeatedly with the beats of the melody or the rhythm.

12. The method as claimed in claim 9, wherein said brain imaging device is a PET device or an SPECT device.
